(19) United States
(12) Patent Application Publication
(10) Pub. No.: US 2024/0115436 A1
(43) Pub. Date: Apr. 11, 2024

(54) FEMININE HYGIENE PAD WITH FOAM ABSORBENT AND RESERVOIR SPACER LAYER

(71) Applicant: The Procter & Gamble Company, Cincinnati, OH (US)

(72) Inventors: Amit Kumar KAUSHIK, West Chester, OH (US); Digvijay RAWAT, Cincinnati, OH (US); Andrea Pfarr SWITZER, Maineville, OH (US)

(21) Appl. No.: 17/962,571

(22) Filed: Oct. 10, 2022

(51) Int. Cl. A61F 13/538; A61F 13/15; A61F 13/472

(52) U.S. Cl. CPC A61F 13/538; A61F 13/15203; A61F 13/472; A61F 2013/15512; A61F 2013/5386; A61F 2013/5395

(57) ABSTRACT

Disclosed is a feminine hygiene pad with an absorbent system including: an absorbent foam layer having a wearer-facing surface and an outward-facing surface, having a plurality of apertures providing z-direction passageways from the wearer-facing surface to the outward-facing surface; and a porous spacer layer beneath the foam layer and underlying a majority of openings of the apertures in an outward-facing surface of the foam layer. The spacer layer functions to provide structure, beneath the absorbent foam layer, that holds open a desired amount of unoccupied space between the absorbent layer and a backsheet of the pad, into which unabsorbed menstrual fluid may flow and temporarily reside, during the time required for the absorbent layer to effectively imbibe the fluid.

### BACKGROUND

Feminine hygiene pads including open-celled foam absorbent materials may be manufactured to have good absorption capacity, relative thinness/low caliper, and a soft, compliant, resilient and cushiony feel—a combination of features that users of feminine hygiene pads value. For some users, however, it may be desired that a pad provide greater acquisition speed, and additionally or alternatively, greater ability to effectively intake a relatively rapid discharge of menstrual fluid, than is currently associated with such pads. Particularly for users who tend to experience relatively heavy menstrual flow, a quantity of menstrual fluid may collect within the vaginal cavity during a period of rest or relative inactivity, and then be expelled suddenly by shifting body movements. For such users, one or both of the enhancements described above can further reduce risk that fluid might escape the pad before the pad can intake and contain it, and soil underwear, outer clothing, bedding, etc. Room for further improvement exists.

### BRIEF DESCRIPTION OF THE DRAWINGS

FIG. 1 is a top side plan view of an example of a non-limiting feminine hygiene pad.

FIGS. 2A and 2B are top side plan views of the feminine hygiene pad like that shown in of FIG. 1, shown with a topsheet layer removed, with two differing, non-limiting examples of aperture patterns.

FIG. 3A is a schematic lateral cross section of the pad of FIGS. 1 and 2A, taken through line 3-3 in FIG. 1.

FIG. 3B is an expanded view of the pad as shown in FIG. 3A.

FIG. 4 is an expanded view of the pad as shown in FIG. 3A, in an alternative non-limiting example.

FIG. 5 is an underside plan view of the feminine hygiene pad of FIG. 1, shown with a backsheet layer removed, and depicting a boundary between a central region and a peripheral region of a spacer layer.

FIGS. 6A-6C are schematic expanded views of non-limiting examples of adhesive deposits within a discharge region.

FIG. 7A is a schematic cross section of a measurement apparatus configuration used in the Permeability Measurement Method described herein, taken through a vertical plane that bisects the depicted fluid vessel 601.

FIG. 7B is a view of the measurement apparatus as illustrated in FIG. 6A, illustrated with added elements in preparation for commencement of a measurement procedure.

FIG. 7C is a view of the measurement apparatus as illustrated in FIG. 6B, illustrated following commencement of a measurement procedure.

FIG. 8A is a perspective view of a sample weight used in the Permeability Measurement Method described herein.

FIG. 8B is a top view of the sample weight depicted in FIG. 7A.

FIG. 8C is a vertical cross section view of the sample weight depicted in FIG. 7A, taken through line B-B shown in FIG. 7B.

FIG. 9 is a top view of a sample support used in the Permeability Measurement Method described herein.

### DESCRIPTION OF EXAMPLES

### Definitions

For purposes herein, the following terms have the following definitions:

An “aperture” is an opening of any x-y planar shape profile, that is deliberately formed, punched or cut along a z-direction entirely through a layer, so as to provide a z-direction passageway through the layer.

“Lateral”—with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface. With respect to an absorbent article, the “x-direction” is also the lateral direction.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above. With respect to an absorbent article, the “y-direction” is also the longitudinal direction.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

“x-y plane,” with reference to an absorbent article such as a feminine hygiene pad, or component thereof, when laid out flat on a horizontal surface, means any horizontal plane occupied by the horizontal surface or any layer of the article or component.

“z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms characterizing relative vertical positioning, when used herein to refer to layers, components or other features of an absorbent article such as a feminine hygiene pad, are relative the z-direction and are to be interpreted with respect to the orientation of the article as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a topsheet has a wearer-facing surface that lies closer to the wearer than the opposite, outward-facing surface of the topsheet.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### DESCRIPTION

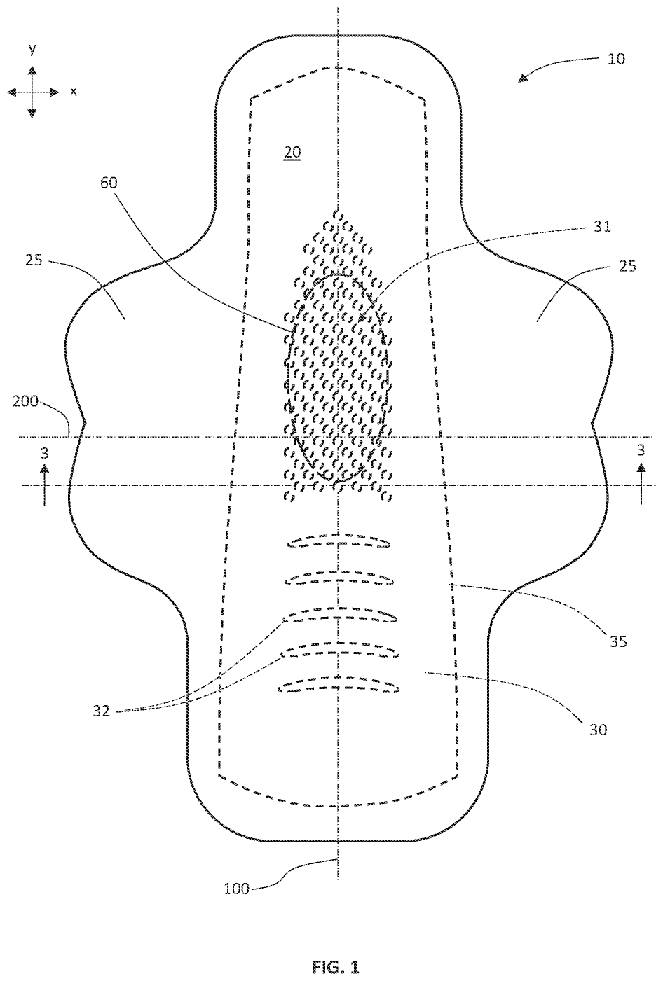

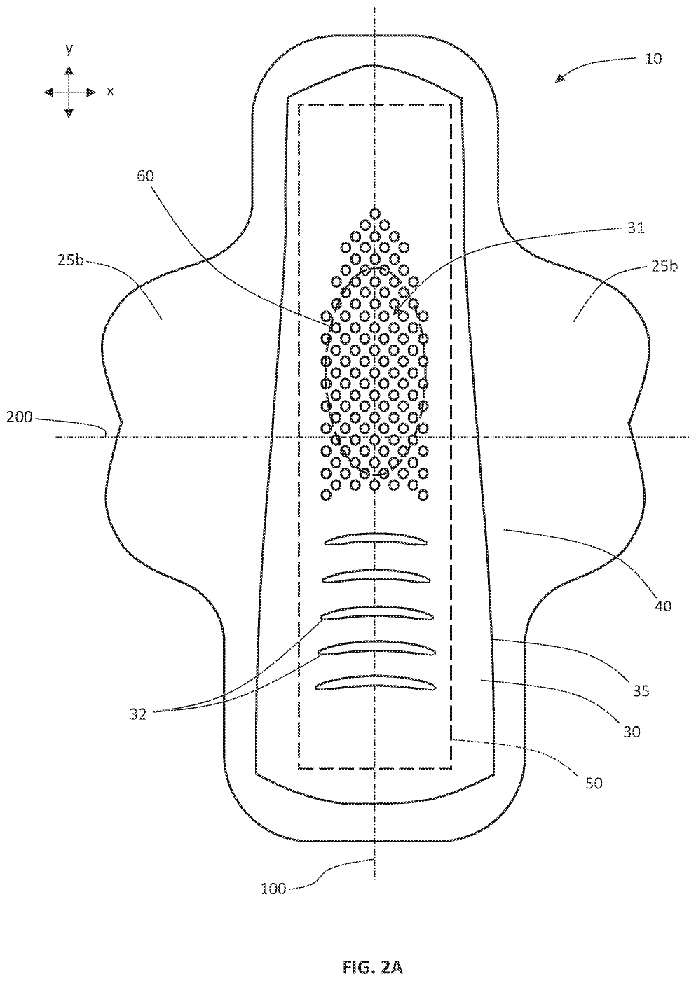

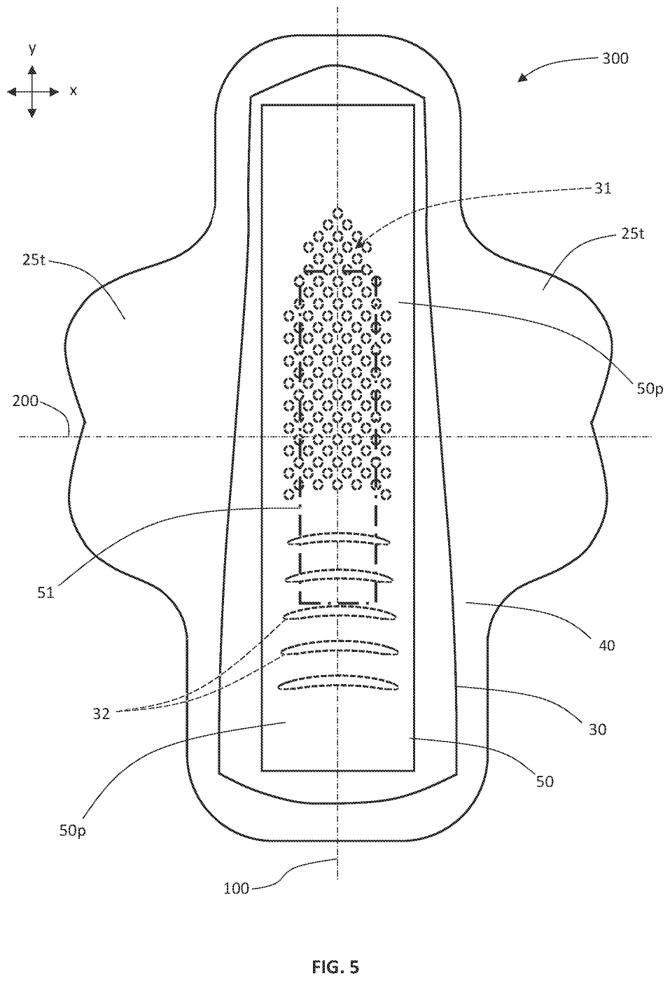

Referring to FIGS. 1, 2 and 3A, a feminine hygiene pad 10 may include a liquid permeable topsheet 20, a liquid impermeable backsheet 40 and an absorbent layer 30 disposed between the topsheet and the backsheet. The absorbent layer has an outer perimeter edge 35. In regions outside the outer perimeter edge 35, the topsheet and the backsheet may be bonded together in laminated fashion by any suitable mechanism including but not limited to adhesive bonding, thermal bonding, pressure bonding, etc., thereby retaining and holding the absorbent layer 30 in place between the topsheet 20 and the backsheet 40. Pad 10 may include opposing wing portions 25 extending laterally outside of perimeter edge 35 by a comparatively greater width dimension than the main portion of the pad. The wing portions 25 may be formed of profiled lateral extensions of the backsheet 25b (shown in FIG. 2) and/or of the topsheet 25t (shown in FIG. 5). The outer surface of the backsheet forming the undersides of the main portion and the wing portions may have deposits of adhesive (not shown) thereon. Such adhesive deposits may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental holding support and helping to protect the leg edges of the underpants against soiling. When pad 10 is packaged, the adhesive deposits may be covered by one or more sheets of release film or paper (not shown) that covers/shields the adhesive deposits from contact with other surfaces until the user is ready to remove the release film or paper and place the pad for use.

Topsheet

The topsheet 20 is positioned adjacent a wearer-facing surface of the absorbent layer 30 and may be joined thereto and to the backsheet 40 by any suitable attachment or bonding method. The topsheet 20 and the backsheet 40 may be joined directly to each other in the peripheral regions outside the perimeter edge 35 of the absorbent layer 30 and may be indirectly joined by directly joining them respectively to wearer-facing and outward-facing surfaces of the absorbent layer or additional optional layers included with the pad.

The topsheet 20 is formed of a singular or laminate material that is, preferably, compliant, soft feeling, and non-irritating to the wearer's skin. A suitable topsheet material will include a liquid pervious material that is comfortable when in contact with the wearer's skin and permits discharged menstrual fluid to rapidly penetrate through it. A suitable topsheet may be made of various materials such as a suitable apertured, liquid permeable film, a liquid permeable nonwoven web material, or a combination or laminate thereof. In some non-limiting examples, the topsheet 20 may be formed of a polymeric film material having apertures therethrough, which are provided to enable fluid movement, from the wearer-facing surface, through the topsheet, to the absorbent material(s) beneath. Such liquid permeable, apertured films are known in the art. They provide a resilient three-dimensional fabric-like structure. Such films have been disclosed in detail, for example, in U.S. Pat. Nos. 3,929,135; 4,151,240; 4,319,868; 4,324,426; 4,343,314; 4,591,523; U.S. Pat. Nos. 4,609,518; 4,629,643; 4,695,422; and WO 96/00548.

In other non-limiting examples, the topsheet 20 may be formed of a liquid permeable nonwoven web material. Examples of nonwoven web materials that may be suitable for use as the topsheet include fibrous materials made from natural fibers, modified natural fibers, synthetic fibers, or combinations thereof. Some suitable examples are described in U.S. Pat. Nos. 4,950,264, 4,988,344; 4,988,345; 3,978,185; 7,785,690; 7,838,099; 5,792,404; and 5,665,452.

In some examples, the topsheet may comprise tufted structures as described in U.S. Pat. Nos. 8,728,049; 7,553,532; 7,172,801; 8,440,286; 7,648,752; and 7,410,683. The topsheet may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535 and US 2015/041640. Another suitable topsheet may be formed from a three-dimensional substrate as detailed in US 2017/0258647. The topsheet may have one or more layers, as described in US 2016/0167334; US 2016/0166443; and US 2017/0258651.

As contemplated herein, component nonwoven web material from which topsheet 20 may be cut may be a nonwoven web material that includes or consists predominately (by weight) of fibers spun from polymeric resin such as polyolefins, including polypropylene, polyethylene and variants, blends, and bicomponent or multicomponent arrangements thereof.

The nonwoven web may be formed via any suitable process by which spun fibers of indefinite lengths may be distributed and accumulated in a controlled fashion onto a moving forming belt to form a batt having a desired distribution of the fibers, to a desired basis weight. Suitable processes may include spunbonding and meltblowing. After accumulation the batt may be processed to consolidate and bond the fibers into a cohesive web by any suitable method, including calendering, calendar thermal bonding, calendar compression bonding, through-air bonding, etc. The consolidated web may be subjected to further processes such as hydroenhancing or hydroentangling, to increase z-direction entanglement of fibers, and increase loft.

In some examples the nonwoven web material may be formed in a co-forming process in which hydrophilic fibers (such as plant-based, e.g., cotton fibers, rayon fibers, etc.) of finite lengths are physically blended or mixed with streams of spun fibers of longer but indefinite lengths, spun from polymeric resin, and laid down on a forming belt to form a web as described in, for example, U.S. Pat. Nos. 8,017,534; 4,100,324; US 2003/0200991; U.S. Pat. No. 5,508,102; US 2003/0211802; EP 0 333 228; WO 2009/10938; US 2017/0000695; US 2017/0002486; U.S. Pat. No. 9,944,047; 2017/0022643 and US 2018/0002848.

Absent enhancements to the materials and/or processes involved, generally, monocomponent fibers spun from polymer resin tend to have relatively simple surface geometry, typically a round or approximately oval-shaped cross section, and a substantially non-curled or non-crimped configuration along the lengths thereof. As a consequence, when the spun fibers are deposited and accumulated on a forming belt, calendered and bonded (e.g. in a spunbonding process), the resulting nonwoven web product will have relatively low loft and a relatively flat appearance, as compared with a web of a comparable basis weight formed of more complexly-shaped, e.g., curled or crimped, fibers. A lower loft nonwoven web may be perceived by some consumers to have a comparatively less pleasing feel and appearance, i.e., it may perceived to be, relatively, not as soft or luxurious, as a higher-loft one.

To add loft to the web without increasing basis weight (and material usage), and to increase opacity of the web, the fibers used to make the web may be spun in multicomponent, e.g., bicomponent fiber configurations. Resin-processing equipment and beams of spinnerets may be configured, and polymer resins may be selected, to spin bicomponent fibers that crimp or curl as they leave the spinnerets as molten polymer streams, and subsequently cool and solidify into fibers. Known processes and polymer resin selections may be used to produce curly spun bicomponent fibers wherein the fibers have side-by-side, eccentric core-sheath, or other non-coaxial polymer component cross-sectional configurations. In such non-coaxial configurations, one of the polymer components may be selected and/or formulated to have a differing melting temperature and/or cooling contraction rate than the other polymer component. Upon cooling, the differing properties of the polymer components and non-coaxial cross-sectional arrangement of component sections of the molten fiber streams impart curl to the fibers as they cool, contract at differing rates and solidify. The respective polymer resin components may be differing polymers, differing forms or variants of the same polymers, or differing blends thereof. More detailed disclosure of spinning curled or crimped bicomponent fibers and forming a nonwoven web thereof may be found in, for example, U.S. Pat. No. 8,501,646; US EP 1 988 793; and US 2007/0275622. In some examples bicomponent fibers may have respective predominately polypropylene-based resin components formulated to impart differing melting temperatures to the respective components. In some examples bicomponent fibers may have respective components in which one component is predominately polypropylene-based, and the other component is predominately polyethylene-based. In some more particular examples the bicomponent fibers may be spun with an eccentric core-sheath component configuration wherein a predominately polypropylene-based component is the core component and a predominately polyethylene-based component is the sheath component; wherein the polypropylene-based component may be desired for its greater tensile strength, and the polyethylene-cased component may be desired for its smoother, more lubricious surface feel, that helps impart a silky feel to the fiber and to the nonwoven web material. It will be appreciated that other combinations of polyolefins and/or other spinnable thermoplastic resins may be selected for their differing cooling contraction rates and other differing qualities that affect the qualities (including curl or crimp) and properties of the spun fibers in differing ways.

A perceivable pillowy, springy loft may be imparted to a topsheet web material using bicomponent fiber constituents, while undesirable pilling may be avoided, by including a layer, which may be a relatively thin layer, of spun monocomponent fibers overlying the layer(s) of bicomponent fibers, at least on the wearer-facing side of the web. On a web-forming line with a moving forming belt, one or more beams configured to spin bicomponent fibers may be disposed upstream of a beam configured to spin monocomponent fibers. In such a configuration the downstream monocomponent fiber-spinning beam is disposed to deposit a layer of spun monocomponent fibers over the previously-deposited bicomponent fibers on the moving belt. Alternatively, a monocomponent fiber spinning beam may be situated upstream of the bicomponent fiber spinning beam(s). In both cases, it is contemplated that a monocomponent fiber layer will serve as the wearer-facing layer of the topsheet in feminine hygiene pad end products. Following formation of a batt of spun fibers having bicomponent fiber and monocomponent fiber layers, the batt may be calender-bonded in the nip between a pair of calender bonding rollers (one or both bearing a pattern of bonding protrusions), forming a pattern of fusion bonds in the web via heat and/or compression, reflecting the pattern of bonding protrusions on the roller. For purposes of maximizing likelihood of effective fusion bonding at each bond site, it may be desired that a polymer resin component of at least one of the component sections of the bicomponent fibers, and the polymer resin component from which the monocomponent fibers are spun, are of like chemistry.

In some circumstances it may be desired that the roller bearing the bonding protrusions be the roller that faces the monocomponent fiber layer in the nip, which may help impart a quilted appearance to the monocomponent/wearer-facing layer—for enhanced perception of pillowy loft in the topsheet. In other circumstances it may be desired that the roller bearing the bonding protrusions be the roller that faces a layer of bicomponent fibers in the nip—which may help improve cohesiveness of the web.

For purposes of preserving the pillowy, lofty appearance of the web material and avoiding imparting an unwanted amount of stiffness to the web resulting from bonding, it may desired that the bonded area ((total area of bonds/total area of web)×100%) be from 8 percent to 20 percent, or more preferably from 10 percent to 16 percent. Bonded area percentage of a bonded nonwoven web material is often understood to be reflected by the sum of areas of the bonding surfaces (sometimes called “lands”) of the bonding protrusions on the calender bonding roller used, versus the total circumferential surface area of the acting portion of the calender bonding roller bearing the pattern of bonding protrusions. Bond area is often specified in drawings used to depict a bonding protrusion pattern and set forth specifications for manufacturing a bonding roller, or may be calculated based on the dimensions and numerical density/roller surface area of the bonding protrusions reflected in such drawings.

In order to ensure that fluid contacting the top (wearer-facing) surface of a hydrophilic topsheet will move suitably rapidly via capillary action in a z-direction to the bottom (outward-facing) surface of the topsheet where it can be drawn into the absorbent layer, it may be important to ensure that the nonwoven web material forming the topsheet has an appropriate weight/volume density, reflecting suitable presence of interstitial passageways among and between the constituent fibers, through which fluid may move within the nonwoven material. A nonwoven with fibers that are consolidated too densely will have insufficient numbers and volume of interstitial passageways, and the nonwoven will obstruct rather than facilitate rapid z-direction fluid movement. On the other hand, a nonwoven with fibers that are not consolidated enough to provide sufficient fiber-to-fiber contact and/or sufficiently small interstitial passageways may provide insufficient potential for wicking in the z-direction via capillary action. For purposes of balancing desires for web loftiness, opacity and mechanical strength, on one hand, versus limitation of caliper and fiber count/web density for purposes of rapid z-direction movement of discharged fluid on the other, it may be desired that the combination bicomponent fiber/monocomponent fiber web be manufactured to a basis weight of 17 gsm (grams per square meter) to 33 gsm, or more preferably 21 gsm to 29 gsm. For purposes of ensuring suitable rapidity of z-directing wicking, it may be preferred that the web be manufactured to have a caliper that is 0.008 mm to 0.014 mm per unit basis weight in gsm. For example, for a web having a basis weight of 25 gsm, it may be preferred that it be manufactured to have a caliper of 0.20 mm (0.008 mm×25) to 0.35 mm (0.014 mm×25). For purposes herein, caliper of a nonwoven web is measured using the Dry Caliper Measurement Method set forth below. It will be appreciated that caliper may be adjusted by the degree of compression applied to the web in calendering, density and bond area of the bonding pattern used, amount of curl or crimp imparted to the bicomponent fibers, etc. The monocomponent fiber layer (wearer-facing layer) may constitute from 10 percent to 70 percent, more preferably from 20 percent to 50 percent, and even more preferably from 25 percent to 45 percent, of the total basis weight of the web.

As noted, formation of a nonwoven web material with spun fibers that include curled bicomponent fibers can help increase opacity of the web as compared with a spunbond web formed only of monocomponent fibers. This is believed to be a result of increased light scattering and diffusion resulting from greater fiber shape complexity and greater web loft. It has been learned that greater opacity is beneficial for purposes contemplated herein because it increases the concealing capability of a topsheet formed of the web material. In addition to incorporation of bicomponent fibers, the manufacturer may also enhance opacity of the web material by including whitening or opacifying additives with the resin(s) from which the monocomponent and/or bicomponent fibers are spun. In some particular examples, the manufacture may include titanium dioxide opacifier/whitener in an amount of up to 1 percent, 2 percent, 3 percent, 4 percent, or even 5 percent, by weight of the resin for any one, two or all of the individual bicomponent fiber components and monocomponent fiber component. For purposes of balancing concealing capability of the web material with basis weight and caliper constraints as discussed herein, it may be desired to adjust basis weight and component resin formulation (including an opacifying additive to an extent deemed useful) to achieve an opacity level for the nonwoven web material of from 30 percent to 42 percent, as measured by the opacity measurement method set forth below.

Many commercially practical thermoplastic resins that may be desired to process and spin into bicomponent fibers are normally hydrophobic. Such resins include polyolefins such as polypropylene and polyethylene. A nonwoven web material formed of such fibers will also be hydrophobic, and as such, will not readily accept or wick aqueous fluid such as menstrual fluid. When such resins are used, therefore, additional measures must be included to render the fibers and/or the nonwoven web hydrophilic. In some examples, a suitable surfactant may be applied to the nonwoven web following its formation. In more particular examples, a suitable surfactant finish used may be SILASTOL PHP 26, a product of Schill+Seilacher GmbH, Boblingen, Germany. The finish may be applied to the web using any suitable method, for example, via kiss roll coater. The finish may be applied in a quantity suitable to impart the nonwoven web with a desired level of hydrophilicity and thereby help impart it with a desired level of capillary absorption/desorption pressure. In particular examples, a finish coating of SILASTOL PHP 26 may be applied in a quantity sufficient to constitute, after drying, surfactant weight quantity that is 0.30 percent to 0.60 percent, more preferably 0.40 percent to 0.50 percent of the basis weight of the nonwoven web material.

Absorbency and wicking performance also may vary according to, and may be manipulated by, the manner in which the web is further processed. Factors such as level of consolidation (i.e., densification) of the fiber mass in the end structure and orientations of the individual fibers within the end structure can affect absorbency and wicking performance.

Thus, for purposes contemplated herein, in combination with being imparted with a suitable basis weight, density and/or caliper as discussed above, it may be desired that nonwoven web material formed in part or in substantial entirety of fibers spun from thermoplastic polymer resin and used to make the topsheet, be formed via a nonwoven web manufacturing process in which substantial portions of the fibers are imparted with directional orientation that includes some z-direction orientation, rather than orientations predominately biased along the machine direction or x-y plane of formation of the web structure. Following any suitable processes in which fibers are distributed and laid down in a batt on a horizontal forming belt (e.g., via a spunbond process), additional process steps that forcibly reorient some of the fibers or portions thereof in the z-direction may be employed. Suitable process steps may include needle punching and hydroentangling or hydroenhancing. Hydroentangling or hydroenhancing, in which an array of fine, high-velocity water jets is directed at the batt as it is conveyed past them, may be desired for its effectiveness in reorienting lengths of fibers while breaking fewer fibers and creating less broken fiber lint and surface fuzz (free fiber ends extending from the surface of the web). A vacuum water removal system (in which air is drawn through the web in a z-direction into a pattern of orifices or pores on a vacuum drum or belt conveying the batt, pulling the jetted water with it) may be desired because it tends to create, add, open and/or clear small z-direction passageways within the fiber matrix of the web, approximately in the pattern of the orifices or pores. Without intending to be bound by theory, it is believed that the portions of the fibers oriented in the z-direction and the z-direction passageways increase the ability and tendency of the web to wick aqueous fluid in the z-direction. In a topsheet, this would mean that the material can more readily wick aqueous fluid from the wearer-facing surface of the topsheet to the outward-facing surface of the topsheet, i.e., down to the absorbent layer below, and may thereby wick fluid less along x-y planar directions (causing a stain from discharged fluid to spread laterally and/or longitudinally).

Absorbent Layer

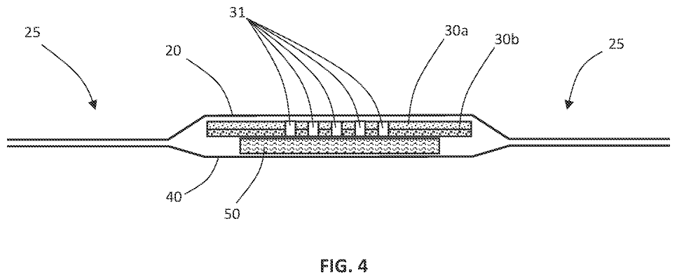

In some examples the absorbent layer 30 may be formed of or include a layer of absorbent open-celled foam material. In some examples, the foam material may include at least first and second sublayers 30a, 30b (FIG. 4) of absorbent open-celled foam material, the sublayers being in direct face-to-face association and contact with each other. In such examples, the wearer-facing sublayer 30a may be a relatively larger-celled foam material, and the outward-facing sublayer 30b may be a relatively smaller-celled foam material, for purposes explained in more detail below.

The open-celled foam material may be a foam material that is manufactured via polymerization of the continuous oil phase of a water-in-oil high internal phase emulsion (“HIPE”).

A water-in-oil HIPE has two phases. One phase is a continuous oil phase comprising monomers to be polymerized, and an emulsifier to help stabilize the HIPE. The oil phase may also include one or more photoinitiators. The monomer component may be included in an amount of from about 80% to about 99%, and in certain examples from about 85% to about 95% by weight of the oil phase. The emulsifier component, which is soluble in the oil phase and suitable for forming a stable water-in-oil emulsion may be included in the oil phase in an amount of from about 1% to about 20% by weight of the oil phase. The emulsion may be formed at an emulsification temperature of from about 20° C. to about 130° C. and in certain examples from about 50° C. to about 100° C.

In general, the monomers may be included in an amount of about 20% to about 97% by weight of the oil phase and may include at least one substantially water-insoluble monofunctional alkyl acrylate or alkyl methacrylate. For example, monomers of this type may include C4-C18 alkyl acrylates and C2-C18 methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40%, and in certain examples from about 10% to about 30%, by weight of the oil phase, a substantially water-insoluble, polyfunctional crosslinking alkyl acrylate or methacrylate. This crosslinking comonomer, or crosslinker, is added to confer strength and resilience to the resulting HIPE foam. Examples of crosslinking monomers of this type comprise monomers containing two or more activated acrylate, methacrylate groups, or combinations thereof. Nonlimiting examples of this group include 1,6-hexanedioldiacrylate, 1,4-butanedioldimethacrylate, trimethylolpropane triacrylate, trimethylolpropane trimethacrylate, 1,1 2-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like. Other examples of crosslinkers contain a mixture of acrylate and methacrylate moieties, such as ethylene glycol acrylate-methacrylate and neopentyl glycol acrylate-methacrylate. The ratio of methacrylate:acrylate group in the mixed crosslinker may be varied from 50:50 to any other ratio as needed.

Any third substantially water-insoluble comonomer may be added to the oil phase in weight percentages of from about 0% to about 15% by weight of the oil phase, in certain examples from about 2% to about 8%, to modify properties of the HIPE foams. In certain cases, “toughening” monomers may be desired to impart toughness to the resulting HIPE foam. These include monomers such as styrene, vinyl chloride, vinylidene chloride, isoprene, and chloroprene. Without being bound by theory, it is believed that such monomers aid in stabilizing the HIPE during polymerization (also known as “curing”) to provide a more homogeneous and better-formed HIPE foam which results in greater toughness, tensile strength, abrasion resistance, and the like. Monomers may also be added to confer flame retardancy, as disclosed, for example, in U.S. Pat. No. 6,160,028. Monomers may be added to impart color (for example vinyl ferrocene); to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam. In some cases, these additional monomers may slow the overall process of conversion of HIPE to HIPE foam, the tradeoff being necessary if the desired property is to be conferred. Thus, such monomers can also be used to slow down the polymerization rate of a HIPE. Examples of monomers of this type comprise styrene and vinyl chloride.

The oil phase may further include an emulsifier to stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate (DGMO), polyglycerol monoisostearate (PGMIS), and polyglycerol monomyristate (PGMM); (b) polyglycerol monoesters of -branched C16-C24 fatty acids, linear unsaturated C16-C22 fatty acids, or linear saturated C12-C14 fatty acids, such as diglycerol monooleate (for example diglycerol monoesters of C18:1 fatty acids), diglycerol monomyristate, diglycerol monoisostearate, and diglycerol monoesters; (c) diglycerol monoaliphatic ethers of -branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate (PGS), which is formed from an alkyl succinate, glycerol, and triglycerol.

Such emulsifiers, and combinations thereof, may be added to the oil phase so that they constitute about 1% to about 20%, in certain examples about 2% to about 15%, and in certain other examples about 3% to about 12%, of the weight of the oil phase. In certain examples, coemulsifiers may also be used to provide additional control of cell size, cell size distribution, and emulsion stability, particularly at higher temperatures, for example greater than about 65° C. Examples of coemulsifiers include phosphatidyl cholines and phosphatidyl choline-containing compositions, aliphatic betaines, long chain C12-C22 dialiphatic quaternary ammonium salts, short chain C1-C4 dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts. In certain examples, ditallow dimethyl ammonium methyl sulfate (DTDMAMS) may be used as a coemulsifier.

Any photoinitiators included may be included at between about 0.05% and about 10%, and in some examples between about 0.2% and about 10% by weight of the oil phase. Lower amounts of photoinitiator allow light to better penetrate the HIPE foam, which can provide for polymerization deeper into the HIPE foam. However, if polymerization is performed in an oxygen-containing environment, it may be desired that there be enough photoinitiator present to initiate the polymerization and overcome oxygen inhibition. Photoinitiators can respond rapidly and efficiently to a light source with the production of radicals, cations, and other species that are capable of initiating a polymerization reaction. Photoinitiators selected for use in forming foams within contemplation of the present disclosure may absorb UV light at wavelengths of about 200 nanometers (nm) to about 800 nm, in certain examples about 250 nm to about 450 nm. If the photoinitiator is in the oil phase, suitable types of oil-soluble photoinitiators include benzyl ketals, α-hydroxyalkyl phenones, α-amino alkyl phenones, and acylphospine oxides. Examples of photoinitiators include 2,4,64trimethylbenzoyldiphosphine]oxide in combination with 2-hydroxy-2-methyl-1-phenylpropan-1-one (50:50 blend of the two is sold by Ciba Speciality Chemicals, Ludwigshafen, Germany as DAROCUR 4265); benzyl dimethyl ketal (sold by Ciba Geigy as IRGACURE 651); α-,α-dimethoxy-α-hydroxy acetophenone (sold by Ciba Speciality Chemicals as DAROCUR 1173); 2-methyl-1-[4-(methyl thio)phenyl]-2-morpholino-propan-1-one (sold by Ciba Speciality Chemicals as IRGACURE 907); 1-hydroxycyclohexyl-phenyl ketone (sold by Ciba Speciality Chemicals as IRGACURE 184); bis(2,4,6-trimethylbenzoyl)-phenylphosphineoxide (sold by Ciba Speciality Chemicals as IRGACURE 819); diethoxyacetophenone, and 4-(2-hydroxyethoxy)phenyl-(2-hydroxy-2-methylpropyl)ketone (sold by Ciba Speciality Chemicals as IRGACURE 2959); and Oligo [2-hydroxy-2-methyl-1-[4-(1-methylvinyl)phenyl]propanone] (sold by Lamberti spa, Gallarate, Italy as ESACURE KIP EM.

The dispersed aqueous phase of a HIPE comprises water, and may also comprise one or more components, such as initiator, photoinitiator, or electrolyte, wherein in certain examples, the one or more components are at least partially water soluble.

One component included in the aqueous phase may be a water-soluble electrolyte. The water phase may contain from about 0.2% to about 40%, in certain examples from about 2% to about 20%, by weight of the aqueous phase of a water-soluble electrolyte. The electrolyte minimizes the tendency of monomers, comonomers, and crosslinkers that are primarily oil soluble to also dissolve in the aqueous phase. Examples of electrolytes include chlorides or sulfates of alkaline earth metals such as calcium or magnesium and chlorides or sulfates of alkali earth metals such as sodium. Such electrolyte can include a buffering agent for the control of pH during the polymerization, including such inorganic counterions as phosphate, borate, and carbonate, and mixtures thereof. Water soluble monomers may also be used in the aqueous phase, examples being acrylic acid and vinyl acetate.

Another component that may be included in the aqueous phase is a water-soluble free-radical initiator. The initiator can be present at up to about 20 mole percent based on the total moles of polymerizable monomers present in the oil phase. In certain examples, the initiator may be included in an amount of from about 0.001 to about 10 mole percent based on the total moles of polymerizable monomers in the oil phase. Suitable initiators include ammonium persulfate, sodium persulfate, potassium persulfate, 2,2′-azobis(N,N-dimethyleneisobutyramidine)dihydrochloride, azo initiators, redox couples like persulfate-bisulfate, persulfate-ascorbic acid, and other suitable redox initiators. In certain examples, to reduce the potential for premature polymerization which may clog the emulsification system, addition of the initiator to the monomer phase may be performed near the end of the emulsification step, or shortly afterward.

Photoinitiator included in the aqueous phase may be at least partially water soluble, and may constitute between about 0.05% and about 10%, and in certain examples between about 0.2% and about 10%, by weight of the oil phase. Lower amounts of photoinitiator allow light to better penetrate the HIPE foam, which can provide for polymerization deeper into the HIPE foam. However, if polymerization is done in an oxygen-containing environment, there should be enough photoinitiator to initiate the polymerization and overcome oxygen inhibition. Photoinitiators can respond rapidly and efficiently to a light source with the production of radicals, cations, and other species that are capable of initiating a polymerization reaction. Photoinitiators selected for use to form foams within contemplation of the present disclosure may absorb UV light at wavelengths of from about 200 nanometers (nm) to about 800 nm, in certain examples from about 200 nm to about 350 nm, and in certain examples from about 350 nm to about 450 nm. If a photoinitiator is to be included in the aqueous phase, suitable types of water-soluble photoinitiators may include benzophenones, benzils, and thioxanthones. Examples of photoinitiators include 2,2′-Azobis[2-(2-imidazolin-2-yl)propane]dihydrochloride; 2,2′-Azobis[2-(2-imidazolin-2-yl)propane]disulfate dehydrate; 2,2′-Azobis(1-imino-1-pyrrolidino-2-ethylpropane)dihydrochloride; 2,2′-Azobis[2-methyl-N-(2-hydroxyethyl)propionamide]; 2,2′-Azobis(2-methylpropionamidine)dihydrochloride; 2,2′-dicarboxymethoxydibenzalacetone, 4,4′-dicarboxymethoxydibenzalacetone, 4,4′-dicarboxymethoxydibenzalcyclohexanone, 4-dimethylamino-4′-carboxymethoxydibenzalacetone; and 4,4′-disulphoxymethoxydibenzalacetone. Other suitable photoinitiators that can be used are listed in U.S. Pat. No. 4,824,765.

In addition to the previously described components other components may be included in either the aqueous or oil phase of a HIPE. Examples include antioxidants, for example hindered phenolics, hindered amine light stabilizers; plasticizers, for example dioctyl phthalate, dinonyl sebacate; flame retardants, for example halogenated hydrocarbons, phosphates, borates, inorganic salts such as antimony trioxide or ammonium phosphate or magnesium hydroxide; dyes and pigments; fluorescers; filler particles, for example starch, titanium dioxide, carbon black, or calcium carbonate; fibers; chain transfer agents; odor absorbers, for example activated carbon particulates; dissolved polymers; dissolved oligomers; and the like.

HIPE foam is produced from the polymerization of the monomers comprising the continuous oil phase of a HIPE. In certain examples, a HIPE foam layer may have one or more sublayers, and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). For example, for a HIPE foam if the difference relates to pore size, the average pore size in the respective sublayers may differ by at least about 20%, in certain examples by at least about 35%, and in still other examples by at least about 50%. In another example, if the differences in the sublayers of a HIPE foam layer relate to density, the densities of the layers may differ by at least about 20%, in certain examples by at least about 35%, and in still other examples by at least about 50%. For instance, if one layer of a HIPE foam has a density of 0.020 g/cc, another layer may have a density of at least about 0.024 g/cc or less than about 0.016 g/cc, in certain examples at least about 0.027 g/cc or less than about 0.013 g/cc, and in still other examples at least about 0.030 g/cc or less than about 0.010 g/cc. If the differences between the layers are related to the chemical composition of the HIPE or HIPE foam, the differences may reflect a relative amount difference in at least one monomer component, for example by at least about 20%, in certain examples by at least about 35%, and in still further examples by at least about 50%. For instance, if one sublayer of a HIPE or HIPE foam is composed of about 10% styrene in its formulation, another sublayer of the HIPE or HIPE foam may be composed of at least about 12%, and in certain examples of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent layer of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from 1 to 200 μm and in certain examples may be less than 100 HIPE foam layers of the present disclosure having two major parallel surfaces may be from about 0.5 to about 10 mm thick, and in certain examples from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed communication with adjoining cells. The cells in such substantially open-celled HIPE foam structures have intercellular openings or windows that are large enough to permit ready fluid transfer from one cell to another within the HIPE foam structure. For purposes of the present disclosure, a HIPE foam is considered “open-celled” if at least about 80% of the cells in the HIPE foam that are at least 11 μm in size are in fluid communication with at least one adjoining cell.

In addition to being open-celled, in certain examples HIPE foams are adapted to be sufficiently hydrophilic to permit the HIPE foam to absorb aqueous fluids. In some examples the internal surfaces of a HIPE foam may be rendered hydrophilic by residual hydrophilizing surfactants or salts left in the HIPE foam following polymerization, by selected post-polymerization HIPE foam treatment procedures (as described hereafter), or combinations of both.

In certain examples, for example when it is used to form an absorbent layer of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In certain examples, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In certain examples, the Tg of this region will be less than about 40° C. for foams used at about ambient temperature conditions; in certain other examples Tg will be less than about 30° C. For HIPE foams used in applications wherein the use temperature is higher or lower than ambient temperature, the Tg of the continuous region may be no more than 10° C. greater than the use temperature, in certain examples the same as use temperature, and in further examples about 10° C. less than use temperature wherein flexibility is desired. Accordingly, monomers are selected as much as possible that provide corresponding polymers having lower Tg's.

HIPE foams useful for forming absorbent layers and/or sublayers within contemplation of the present disclosure, and methods for their manufacture, also include but are not necessarily limited to those foams and methods described in U.S. Pat. Nos. 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711;U.S. Pat. Nos. 5,397,316; 5,331,015; 5,292,777; 5,268,224; 5,260,345; 5,250,576; 5,149,720; 5,147,345; and US 2005/0197414; US 2005/0197415; US 2011/0160326; US 2011/0159135; US 2011/0159206; US 2011/0160321; and US 2011/0160689, which are incorporated herein by reference to the extent not inconsistent herewith.

Absorbent Layer Apertures

As reflected in FIGS. 1, 2 and 3A, an absorbent layer 30 formed of foam may include one or more patterns of apertures 31, 32, including at least a first pattern disposed within an expected discharge region 60. As identified for purposes herein, the discharge region 60 is coincident with (but not necessarily longitudinally centered about) the lateral axis 200 of the pad; is substantially centered about the longitudinal axis 100 of the pad; and has an elliptical outline with a major longitudinal axis 5 cm long and a minor lateral axis 2.5 cm wide. Apertures 31, 32 may be punched, cut or otherwise formed through the entire z-direction depth of the foam absorbent layer. When a foam absorbent layer is disposed in direct contact with a topsheet as described herein, with no intervening acquisition layer formed of another material, apertures 31, 32 may serve as a group of small reservoirs to receive, temporarily hold, and aid in distributing rapid discharges of relatively small quantities of menstrual fluid, until the foam has sufficient time to distribute and absorb the fluid via capillary action. Alternatively or additionally, the apertures may serve as passageways to channel fluid, received in a sudden discharge, through the absorbent layer down to a spacer layer below, as described herein, which provides additional reservoir volume along with fluid distribution capability. Additionally, such apertures help decrease bending stiffness of the absorbent layer 30, which may help increase comfort of the pad for the wearer. To provide passageways through which fluid will readily flow, a pattern of round, oval, or stadium-shaped apertures having an average diameter or other largest dimension of 2.0 mm to 4.0 mm and average x-y plane opening area on the wearer-facing surface of 3 mm² to 13 mm², and more preferably 2.5 mm to 3.5 mm with average x-y plane opening area on the wearer-facing surface of 5 mm² to 10 mm², of the absorbent layer 30, may be included, within, for example, in the area occupied by the discharge region 60. For a good balance between providing a sufficient number of passageways without removing too much absorbent material from the absorbent layer 30 (taking away absorption capacity), it may be desired that the apertures within the discharge region 60, have a numerical density of 3.0 to 9.0 apertures per cm², more preferably 4.0 to 8.0 apertures per cm², or even more preferably 5.0 to 7.0 apertures per cm², surface area of the wearer-facing surface of the absorbent layer 30. In selecting the appropriate average size, numerical density, and surface area occupied by the pattern of apertures, the manufacturer may wish to balance the volume of the reservoirs or passageways desired with the need to retain absorbent material in locations proximate to and about the expected discharge location. Additional details concerning configurations of such apertures in combination with examples of suitable absorbent layers may be found in U.S. Pat. No. 8,211,078.

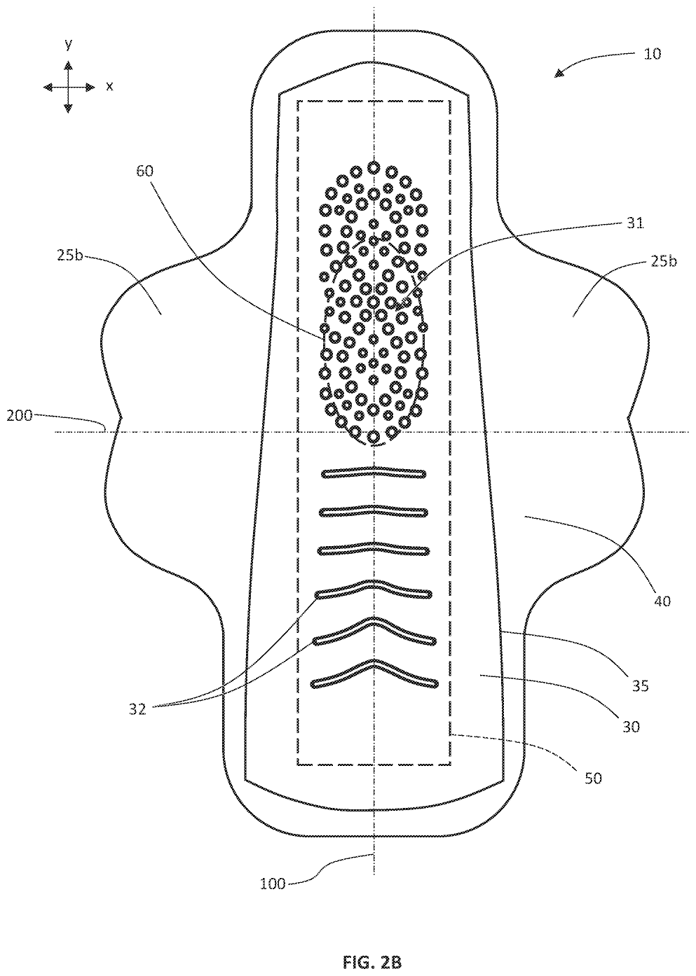

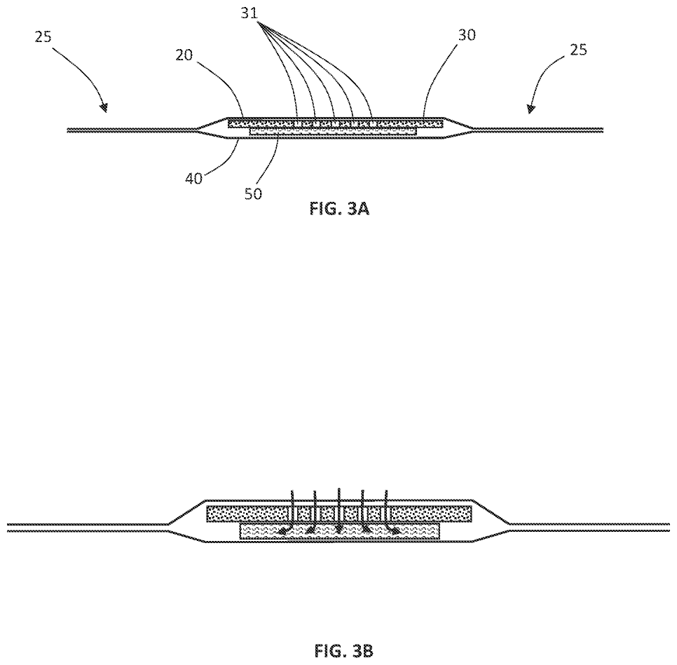

As suggested in FIG. 3B, a pattern of apertures 30, 31 will enable the pad and the foam absorbent layer 30 to more readily accept a sudden discharge of fluid, and enable it to pass quickly through the absorbent layer down to the spacer layer, and reside within the spacer layer during the time required for the absorbent layer to imbibe and absorb the fluid.

Interface Between Topsheet and Absorbent Layer

In examples in which the topsheet is formed of a hydrophilic and/or absorbent web material, the topsheet material may tend to retain fluid on its wearer-facing and outward-facing surfaces, and within the interstitial spaces between and along the surfaces of the fibers of the web material, unless the underlying material has absorption capacity and absorption pressure greater than the desorption pressure of the topsheet, and there is sufficient direct contact maintained between the topsheet and the underlying absorbent layer to enable the fluid to move from fiber surfaces within the topsheet structure, directly to surfaces of material within the underlying absorbent layer structure, such that the underlying absorbent layer may draw the fluid from the topsheet. Prior to the time it is fully saturated, an absorbent material will not release absorbed fluid unless an adjacent material with greater affinity for the fluid is in sufficient direct contact. Accordingly, it is important to provide structure sufficient to maintain sufficient contact, without obstructing fluid movement. No intervening layer or structure of material, or at least no intervening layer or structure of material less absorbent that that of the absorbent layer, should be interposed between the material of the topsheet 20 and the material of the absorbent layer 30, at least within the discharge region 60, more preferably over a majority of the wearer-facing surface area of the absorbent layer 30, and even more preferably over the entirety of the wearer-facing surface area of the absorbent layer 30—unlike systems provided in many current feminine hygiene pads, which include a distinct fluid acquisition/distribution material layer between the topsheet and the absorbent materials of the absorbent core.

In some examples, sufficient direct contact between the topsheet 20 and the absorbent layer 30 may be effected by deposit(s) of adhesive between the topsheet and the absorbent layer, adhesively bonding them in close z-direction proximity. The adhesive may be applied in a pattern or arrangement of adhesive deposits interspersed with areas in which no adhesive is present (unbonded areas), such that the adhesive holds the two layers in close z-direction proximity, while areas remain in which no adhesive is present to obstruct z-direction fluid movement between the layers.

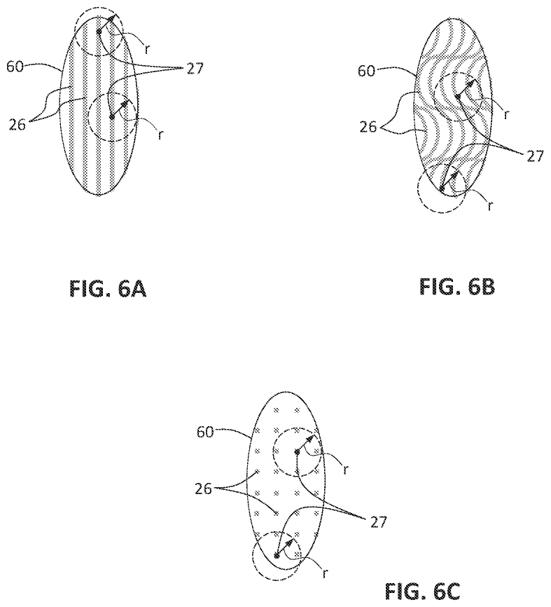

Referring to FIGS. 1, 2 and 6A-6C, to ensure that the topsheet and absorbent layer are held in sufficiently close z-direction proximity at least in the area of the topsheet expected to receive a discharge of fluid, it may be desired to dispose a discharge region 60 on the pad at a location that includes the intersection of the longitudinal and lateral axes 100, 200. The discharge region 60 should be of sufficient size to be reliably present beneath the expected discharge location when the pad is in use, with reasonably minor variations of placement by the wearer within the underpants; accordingly, it may be desired that the discharge region have an area of at least 15 cm², more preferably at least 30 cm². Even more preferably, it may be desired that the discharge region have an area that is at least half of the total wearer-facing surface area of the absorbent layer. (Note: FIGS. 6A-6C are not presented herein as actual size or scale depictions.)

To ensure that the topsheet 20 and the absorbent layer remain in sufficient z-direction proximity during use, it may be desired that, within any identifiable first point location 27 within the discharge region, at which the topsheet is bonded to the absorbent layer, there is a second point location at which the topsheet is bonded to the absorbent layer, within a 10 mm radius, more preferably within a 6 mm radius, 5 mm radius, 4 mm radius, and even more preferably with a 3 mm radius r of the first point location. Referring to FIGS. 3A-3C, depicting three non-limiting examples, it can be seen that a variety of patterns or arrangements of bonds (via adhesive deposits 26 or other bonding mechanisms) may be employed to impart this feature. Within radius r of each point location 27, there are a number of additional point locations where bonding between the topsheet and the absorbent layer is present in the examples depicted.

It will be appreciated that a continuous deposit of adhesive may be applied to bond the topsheet and the absorbent layer within the entirety of discharge region 60, but that such a continuous deposit of adhesive could form a barrier that would obstruct the movement of fluid from the topsheet to the absorbent layer. Accordingly, it is preferable that, in examples in which the bonding mechanism is deposits of adhesive, the deposits are disposed in a pattern or arrangement that is discontinuous or intermittent such that it creates bonded areas interspersed with unbonded areas between the topsheet and the absorbent layer. Additionally, when the absorbent layer is formed of an open-celled foam (such as a foam contemplated herein) it may be desired that the adhesive selected not effect adhesion to the absorbent layer via chemical, dispersive or diffusive adhesion with the foam layer at the adhesive deposit locations, but rather, that it effect adhesion to the foam layer mechanically, by flowing to a limited extent into the cells, at least partially assuming the shapes thereof, and solidifying in such position to form mechanical interlocks with the cell structures, which enable the adhesive to hold the topsheet to the absorbent layer. Such an adhesive may be preferred so as not to alter the molecular structure or composition of the foam material, potentially negatively affecting its fluid absorption properties or mechanical strength. In one example, a suitable adhesive for use with a HIPE foam may be H1750 hot melt adhesive from Bostik, Wauwatosa, Wisconsin (currently a subsidiary of Arkema, Columbes, France).

Unapertured topsheets for feminine hygiene pads formed of nonwoven web material and including or consisting predominately of hydrophilic fibers are known and have been included with some feminine hygiene products to date. (Herein, an “unapertured” nonwoven topsheet is one in which a majority of its surface area has not been subjected to any process that creates an arrangement of holes or apertures entirely therethrough, that persist prior to wetting of the topsheet, of an average size (greatest dimension) greater than 0.5 mm along an x-y planar direction.) Although favored by some consumers for their pleasant feel against the skin, topsheets formed of hydrophilic nonwoven web material have been disfavored by other consumers as a result of their substantial absorbency, i.e., capillary absorption and desorption pressures, causing them to resist drainage by conventionally included acquisition/distribution and absorbent layer structures. Following a discharge of menstrual fluid, a pad with such a topsheet overlying a conventional absorbent structure can feel to the user like a wet cloth held against the skin for an extended time period, which many users find objectionable.

However, an unapertured hydrophilic fiber topsheet overlaid in direct, sufficient face-to-face proximate relationship with a foam absorbent layer or other layer adapted/manufactured to have capillary absorption capability sufficient to draw fluid from the topsheet, without any intervening less absorbent layers and in combination with other structural features as described herein, will be substantially drained of fluid by the absorbent layer, and regain a much drier feel against the skin following a discharge. A suitably composed and manufactured HIPE foam absorbent layer as described herein, for example, has a greater affinity for menstrual fluid than such a topsheet, and thereby, has the capability to draw and retain fluid away from the topsheet when the two are disposed and held in sufficiently effective proximate, contacting relationship with each other. When the absorbent layer has a sufficient volume, it can serve this function over a reasonably suitable time of use of the pad.

Spacer Layer

Referring to FIGS. 1, 2, 3A and 3B, the inclusion of a spacer layer 50 is contemplated herein. The primary purpose of the spacer layer is to provide structure, beneath the absorbent layer 30, that holds open a desired amount of unoccupied space (i.e., a reservoir) between the absorbent layer 30 and the backsheet 40, into which unabsorbed menstrual fluid may flow and temporarily reside, during the time required for absorbent layer 30 to effectively imbibe the fluid. It is desired that spacer layer 50 be disposed beneath, rather than above, the absorbent layer 30, to help prevent unabsorbed fluid from rewetting the topsheet. It is also desired that the absorbent layer 30 include apertures 31, 32 entirely therethrough, i.e., from the wearer-facing surface to the outward-facing surface, as described above, to provide pathways for unabsorbent fluid to move quickly down to the spacer layer.

It is contemplated herein that the spacer layer 50 may be formed entirely of, or alternatively, include, an open-celled foam having a composition differing from, and average cell size greater than, that of a foam absorbent layer. In some examples, the spacer layer may be formed of or include a layer of open-celled polyurethane foam.

It is also contemplated herein that the spacer layer 50 may be formed entirely of, or alternatively, include, a nonwoven batt, web or bundle (herein, collectively, “fiber collection”), formed of filaments or fibers (collectively, fibers).

Where included, it may be desired that such a fiber collection be formed predominantly if not substantially entirely of fibers or filaments spun from one or more thermoplastic polymeric resins, from spinnerets configured to produce fibers having substantially round cross sections.

Such fibers or filaments are not ordinarily absorbent because they generally do not include complex surface geometry, pores or internal spaces into which fluid may flow and be retained.

It may be desired that the thermoplastic resins include one or more of polypropylene (PP), polyethylene (PE) and polyethylene terephthalate (PET). PET may be particularly desired because fibers spun from it have relatively greater stiffness per unit diameter or cross section dimension than fibers spun from other polymers, that are relatively abundant in supply and cost efficient for purposes herein.

In some examples, it may be desired that a predominant portion of the the fibers be bicomponent fibers, and in some examples, it may be desired that the bicomponent fibers are crimped or curled bicomponent fibers. Crimped or curled bicomponent fibers may result from a combination of polymer fiber components having differing melting temperatures and/or contraction rates upon cooling, arranged in a side-by-side or “pie slice” component cross section configuration, or from an eccentric component cross section, for example, an eccentric sheath-core configuration. By their geometry, crimped or curled fibers impart loft and volume to the resulting nonwoven material, and accordingly, their inclusion may increase the volume of open inter-fiber/interstitial space within the material per unit caliper thereof. Accordingly, in some examples, spacer layer 50 may be formed of or include a fiber collection that is predominantly formed of or includes, predominantly, curled bicomponent fibers. In some examples the components of the bicomponent fibers may two differing PE compositions. In some examples the components may be PE and PP; and in some examples the components may be PE and PET, for example, a fiber having a PET core component and a PE sheath component. This particular combination enables the fiber collection to be strengthened and/or stiffened to better retain volume/loft, via fiber-to-fiber thermal bonding effected by, e.g., heating in an oven or hot air-through treatment. The lower melting temperature of the PE enables fusing of the PE sheaths of adjacent fibers, without melting the PET cores, due to their higher melting temperature. This type of fiber-to-fiber bonding may be effected without compressing the fiber collection, which can reduce loft/volume, as occurs in thermal/compression localized spot bonding or calender bonding.

For the purpose of striking a balance between stiffness and resilience for maintaining loft and volume, versus pliability and wearer comfort retained by the pad as a whole, it may be desired that the fiber constituents of the fiber collection have an average denier of 1 to 6, more preferably 1.5 to 5.5, and even more preferably 2 to 5.

It may be desired that a predominant portion, substantially all, or all, of fibers or filaments from which the spacer layer 50 may be formed be spun from inherently hydrophilic material, or alternatively, be treated so as to be rendered hydrophilic, as described above. The purpose is to enable the spacer layer 50 to readily accept fluid moving downwardly through and out apertures 31, 32, and wick or transport the fluid through and across the structure of the spacer layer, thereby distributing it along the underside of the absorbent layer 30 to maximize exposure of its surface area to the fluid, and resulting effective and efficient usage of the absorbent layer. If the spacer layer is predominantly constituted of material(s) having hydrophobic surfaces, it might not readily accept or transport fluid emerging from the apertures at the underside of the absorbent layer.

Based upon the volume of fluid reservoir space desired, it may be preferred that the spacer layer 50 have a void volume of at least 1,000 mm³. (For purposes herein, void volume is calculated by multiplying the wearer-facing surface area of the spacer layer by its dry caliper, minus the mass of each polymer present in the spacer layer divided by its density.) Dry caliper is measured by steps described in the Compression Recovery and Dry Caliper Measurement Methods set forth below. To avoid imparting a level of caliper to the entire assembled product that wearers may find objectionable, however, it may be desired that the dry caliper of the spacer layer be no greater than 3.0 mm, more preferably no greater than 2.0 mm, even more preferably no greater than 1.5 mm, and still more preferably no greater than 1.0 mm.

Relating to void volume, and for purposes of ensuring that the spacer layer 50 will readily accept a gush of fluid from the apertures 30, 31, it may be desirable that the spacer layer be manufactured so as to have a permeability that is no less than a minimum value. Generally, “permeability” reflects the amount of resistance the material presents to the pressurized/forced passage or flow of liquid therethrough. Relatively higher permeability reflects relatively lower resistance to fluid flow, and relatively lower permeability reflects relatively higher resistance to fluid flow. Two materials having the same void volume per unit overall volume may have differing permeability; the level of permeability of a material is substantially affected, in part, by the amount of solid material surface area over/across which fluid must pass to move through the material, and the frictional resistance to fluid movement over the surfaces. Thus, for example, a first nonwoven web material constituted of more numerous, smaller fibers, will exhibit lower permeability, than a second nonwoven web material of the same basis weight and void volume, constituted by fibers of the same composition, but wherein the fibers are larger in size and fewer in number, because the second material will have less fiber surface area.

Thus, for purposes herein, it may be desired that the spacer layer 50 be manufactured so as to have a permeability of at least 1,000 Darcys, preferably at least 3,000 Darcys, and more preferably at least 5,000 Darcys, or 1,000 Darcys to 6,000 Darcys or other upper limits of the particular material(s) used to constitute the spacer layer. All subranges within this greater range are contemplated herein. For purposes contemplated herein, permeability is measured using the Permeability Measurement Method set forth below. In one example, a nonwoven web material deemed suitable to constitute a spacer layer for purposes herein is a carded staple fiber nonwoven web material having a basis weight of 15 gsm, a caliper of about 0.8 mm at 0.1 psi pressure applied in the z-direction, wherein the fibers have an average denier of 6, and consist of bicomponent fibers spun in a sheath-core configuration, wherein the sheath component is formed of PE and the core component is formed of PET, in a weight ratio of about 50:50. This material was found to have a permeability of approximately 5,083 Darcys.

Additionally, it may be desirable that the spacer layer 50 be manufactured so as to have a minimum level of compression recovery. Compression recovery reflects the resiliency of the material, and its relative ability to maintain its caliper and void volume, following compression which may occur, e.g., when a user/wearer sits. Good compression recovery also may be perceived by users as soft and cushiony. Compression recovery may be affected by selection of material(s) to constitute the spacer layer. Where, in some examples, the spacer layer is constituted of a fibrous nonwoven web material, selection of relatively resilient fiber components can help impart good compression recovery. For purposes herein, it may be desired that the spacer layer be manufactured so as to exhibit a compression recovery of at least 75 percent of its caliper to limits within feasibility of the particular material(s) selected to constitute the spacer layer, in application of the Compression Recovery Measurement Method set forth herein.

Preferably the spacer layer will underlie a majority, and more preferably all, of the outward-facing surface openings of apertures 31, 32 present in the absorbent layer 30. This is to ensure that unabsorbed fluid that drains through the apertures is captured by the spacer layer, rather than having pathways to open space between the topsheet and backsheet, which could increase the risk of rewetting the topsheet. At the same time, it may be preferred that the spacer layer not have any portions that extend beyond the perimeter edge of the absorbent layer. This is to ensure that all of the wearer-facing surface of the spacer layer 50 faces an outward-facing surface of the absorbent layer 30, such that fluid within the spacer layer 50 may be more readily absorbed by the absorbent layer 30.

Accordingly, it may be desired that the spacer layer 50 have an outer perimeter that substantially matches that of the absorbent layer 30. Noting, however, that a foam absorbent layer 30 and a spacer layer 50 may be produced by substantially differing manufacturing techniques, it may be desired that spacer layer 50 have an outer perimeter that lies laterally and longitudinally within the outer perimeter of absorbent layer 30. In same examples, for purposes of manufacturing efficiency, a spacer layer 50 may have an outer perimeter that is substantially rectangular, as suggested in FIGS. 2A, 2B and 5. As also suggested in those figures, however, spacer layer 50 may be sized, shaped and positioned to completely underlie all of apertures 31, 32 in/through absorbent layer 30.

Particularly when the spacer layer 50 is imparted with size and surface area that is smaller than that of absorbent layer 30 as suggested in the figures, it may be desired to adhere or bond the spacer layer in place relative the absorbent layer to keep it in its intended position beneath the apertures. In some examples, the spacer layer 50 may be adhered directly to the underside (outward-facing side) of the absorbent layer 30. This may be effected by a deposit or pattern of deposits of adhesive disposed between the layers 30, 50. To ensure that the fluid passageways provided by the apertures 31, 32, to the spacer layer 50 are not obstructed by such adhesive deposits in the expected fluid discharge location, it may be desired to locate the adhesive predominantly about a peripheral region 50p of the spacer layer, i.e., outside boundary 51 (shown in FIG. 5). Boundary 51 equally divides the distance of the perimeter edge(s) of the spacer layer 30 from the intersection of longitudinal axis 100 and lateral axis 200.

Alternatively or in addition, the spacer layer 50 may be adhered to the backsheet 40 by a deposit or pattern of deposits of adhesive disposed between the layers 50, 40. In this position, such adhesive will not obstruct fluid flow from the apertures 31, 32 into the spacer layer. On the other hand, adhering the spacer layer 50 directly to the absorbent layer 30 via deposits of adhesive as described above can help ensure that these layers remain in close proximity or even surface-to-surface contact through shifting wearer body movements, providing better assurance that fluid can move readily into the spacer layer from the apertures 31, 32.

Backsheet

The backsheet 40 may be positioned beneath or subjacent an outward-facing surface of the spacer layer 50 and may be joined thereto by any suitable attachment methods. For example, the backsheet 40 may be secured to the spacer layer 50 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent layer 30 is not joined directly to the backsheet 40.

The backsheet 40 may manufactured to be impermeable to liquids (e.g., urine, menstrual fluid) under ordinary conditions of use of a feminine hygiene pad contemplated herein, and may be manufactured from a plastic film, although other flexible liquid impervious materials may also be used. The backsheet 40 may prevent, or at least substantially inhibit, fluids absorbed and contained within the absorbent layer 30 from escaping and reaching articles of the wearer's clothing which may contact the pad 10, such as underpants, outer clothing, bedding, etc. However, in some instances, the backsheet 40 may be made and/or adapted to permit vapor to pass therethrough (i.e., the backsheet may be made to be breathable), while in other instances the backsheet 40 may be made so as not to permit vapors to pass therethrough (i.e., it is made to be non-breathable). Thus, the backsheet 40 may comprise a polymeric film such as thermoplastic films of polyethylene or polypropylene. A suitable material for the backsheet 40 is a thermoplastic film having a thickness of from about 0.012 mm (0.5 mil) to about 0.051 mm (2.0 mils), for example. Any suitable liquid impermeable backsheet material known in the art is contemplated herein.

Some suitable examples of backsheet materials are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523, 3,989,867, 3,156,242; WO 97/24097; U.S. Pat. Nos. 6,623,464; 6,664,439 and 6,436,508.

The backsheet may be constituted of two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489, 4,341,216, 4,713,068, 4,818,600; EP 203 821, EP 710 471; EP 710 472, and EP 0 793 952.

Permeability Measurement Method

This method enables calculation of permeability of a material (in Darcys) via measurement of the downward movement of test fluid through a test specimen along the z-direction (plumb direction), over a range of falling hydro head indicated by decreasing height of a test fluid in a vessel. The decreasing height of the test fluid inside the vessel, as the fluid drains from the bottom of the vessel through the test specimen, is iteratively measured over time during the procedure. From the collected data together with relevant dimensions of portions of the apparatus through which the fluid moves, the measured wet caliper of the test specimen, and constants associated with gravity and properties of the test fluid chosen, flow rate and permeability may be calculated. All measurements are performed in a laboratory maintained at 23° C.±2 C.° and 50%±2% relative humidity and test specimens are conditioned in this environment for at least 2 hours prior to testing.

Apparatus Components

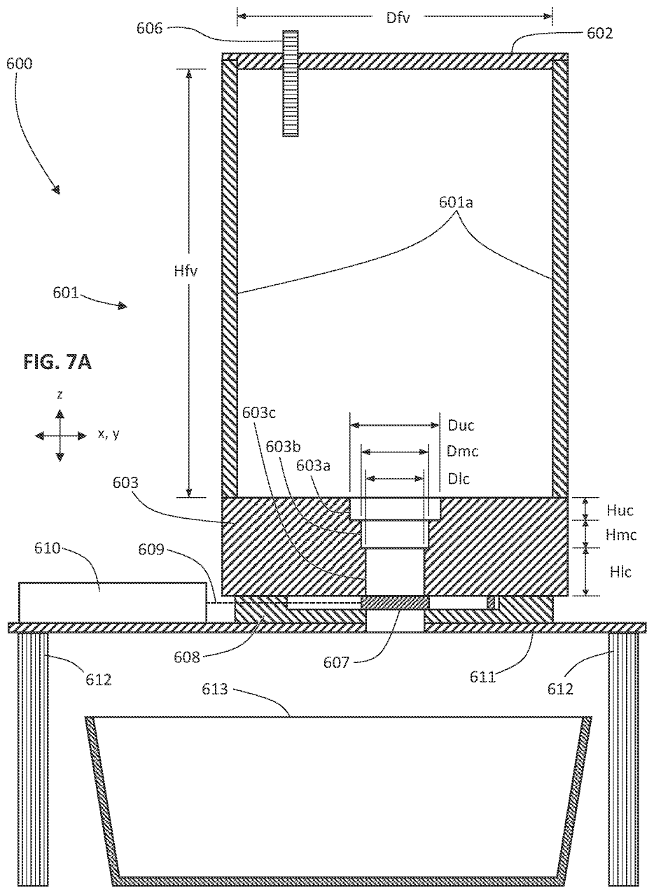

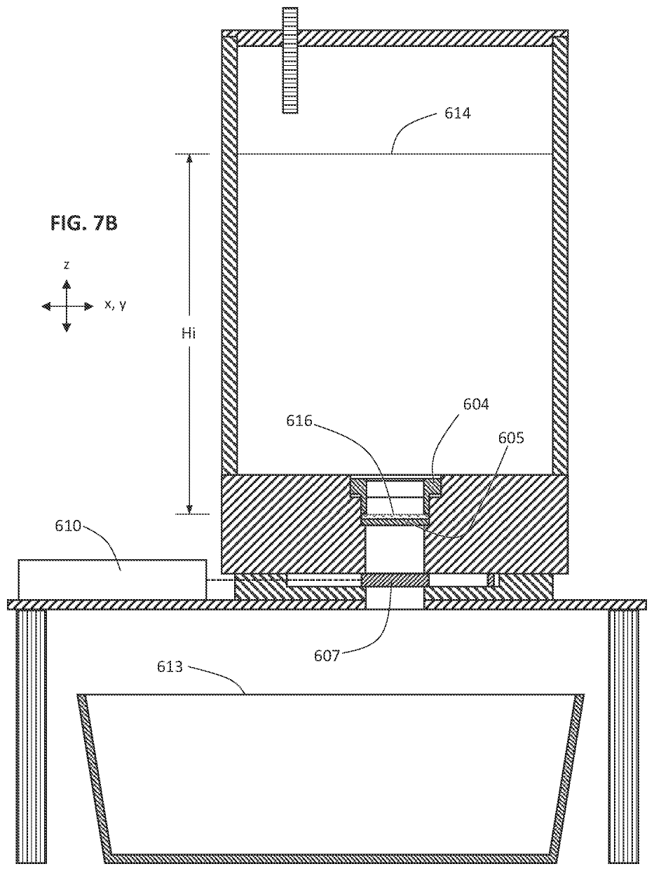

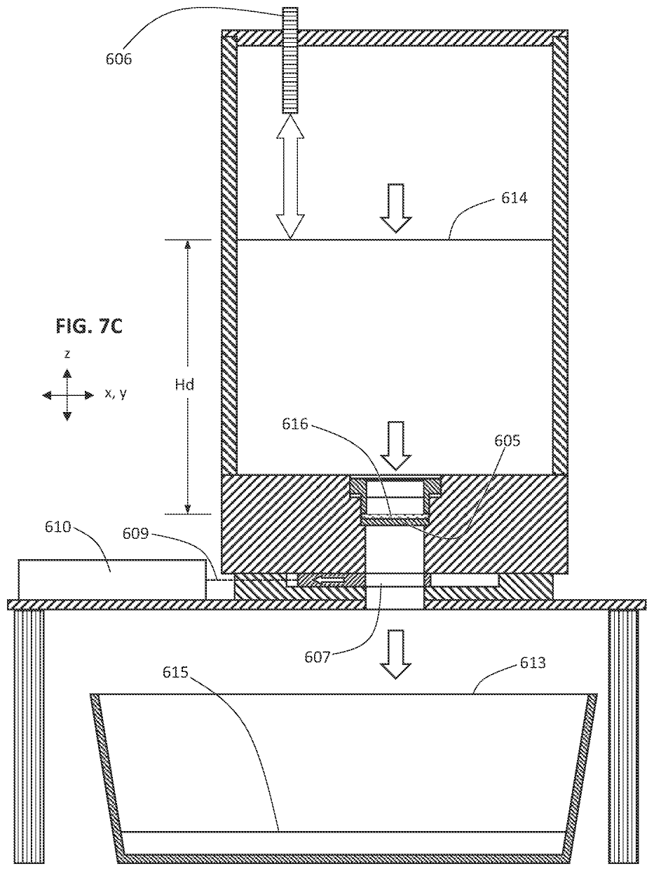

The measurement apparatus 600 and its components are depicted in FIGS. 7A through 9. Referring to FIG. 7A, the apparatus 600 includes a cylindrical fluid vessel 601 including a cylindrical wall 601a with a fitted lid 602 and a base 603 that is sealingly fitted to the bottom of the wall 601a to form fluid vessel 601; a fluid height sensor 606 fitted in and through lid 602; a valve 607 housed in a valve body 608, and a valve actuator 610 mechanically associated with the valve via a linkage 609.

The cylindrical wall has an inside height to the bottom of the lid, Hfv, of 200 mm, an inside diameter of 3⅞ inches (98.425 mm), a wall thickness of ⅜ inch (9.525 mm), and an outer diameter of 4⅝ inches (117.48 mm). The lid 602 is suitably fitted to rest stably on top of the cylindrical wall, but it should not be sealingly fitted thereon; one or more vent holes (not shown) are drilled therethrough to prevent development of negative pressure/vacuum within the fluid vessel as test fluid drains therefrom. The purpose of the lid 602 is to hold and suspend fluid height sensor 606 over the test fluid surface, not to seal the vessel at the top.

Still referring to FIG. 7A, the base 603 has planar, parallel upper and lower surfaces and the upper surface is sealingly affixed to the bottom of the wall 601a. Base 603 is suitably formed or machined to define therewithin a sample chamber having a cylindrical upper chamber portion 603a, a cylindrical middle chamber portion 603b, and a cylindrical lower chamber portion 603c. The three cylindrical chamber portions are coaxial along the vertical/z-direction.

The heights and inner diameters of the three chamber portions are as follows:
Upper chamber portion 603a height Huc: 9.5 mm;
Upper chamber portion 603a inner diameter Duc: 40 mm;
Middle chamber portion 603b height Hmc: 12.5 mm;
Middle chamber portion 603b inner diameter Dmc: 30 mm;
Lower chamber portion 603c height Hlc: 20 mm; and
Lower chamber portion 603a inner diameter Dlc: 26 mm.

Valve body 608 with valve 607 are mounted to the underside of base 603, beneath the lower open end of lower chamber 603c. Valve 607 is configured to be rapidly actuated between fully closed and fully open positions, wherein in the open position, the entirety of lower chamber portion 603c is open to allow fluid to move freely downwardly therefrom without any restriction by valve 607. Valve 607 may be a flat horizontally sliding member, having a circular opening port therethrough, of a diameter of at least 26.0 mm, that is linearly moved to position beneath lower chamber portion 603c upon actuation to the opened position. Alternatively, valve 607 and valve body 608 may have any other suitable configuration adapted to move rapidly between fully closed and fully open positions, wherein when in the fully open position the valve does not present any obstruction to fluid flow downwardly and out from the lower open end of lower chamber portion 603c. Valve 607 and actuator 610 are configured to effect actuation from a fully closed to a fully open position, and vice versa, within no greater than 10 milliseconds for either movement. Actuator 610 may include a solenoid or any other suitable mechanism adapted for this purpose.

Cylindrical wall 601a, lid 602, base 603, and optionally valve body 608 and valve 607, are fabricated of and machined from polished, clear cast acrylic plastic (poly(methyl methacrylate) (PMMA)) stock (known brands include but are not limited to PLEXIGLAS and LUCITE), which may be obtained in various pre-cast tube, rod/bar, disc, sheet and block forms from various suppliers of such materials, such as McMaster-Carr Supply Company (Elmhurst, Illinois). For tube stock used to form wall 601a, tube stock of an inner diameter Dfv varying slightly from that specified herein may be selected, according to availability; in such event, it will be recognized that the corresponding value for the radius r of the fluid vessel, in the equations below, is to be changed to reflect the actual diameter Dfv of the tube stock used.

Fluid height sensor 606 is an ultrasonic height sensor, such as an ML Series part #098-10060, a continuous transmitter through air with an accuracy of about +0.2 mm (TE Connectivity, Schaffhausen, Switzerland and Berwyn, Pennsylvania, USA) or equivalent, interfaced to a computer running software capable of collecting fluid height versus time data throughout the test at a rate of 100 Hz. The fluid height sensor 606 continuously transmits a signal indicating the height of the test fluid within the fluid vessel 601 during the measurement procedure.

The apparatus further includes a support structure, which may include a support platform 611 and height-adjustable legs 612, or any other suitable support structure, configured to stably hold the vessel and valve assembly over a collection vessel 613, with the longitudinal axis of cylindrical wall 601a vertical/plumb and bottom of base 603 level. Where included, a support platform 611 must include an opening or otherwise be configured so as not to obstruct the lower end of lower chamber portion 603c or the fluid exit from valve and valve body 607, 608.

The measurement apparatus further includes a collection vessel 613, of any suitable shape, size and material composition suitable to receive and stably contain the entirety of the volume of test fluid that is used in this method, and fit easily beneath the support structure.

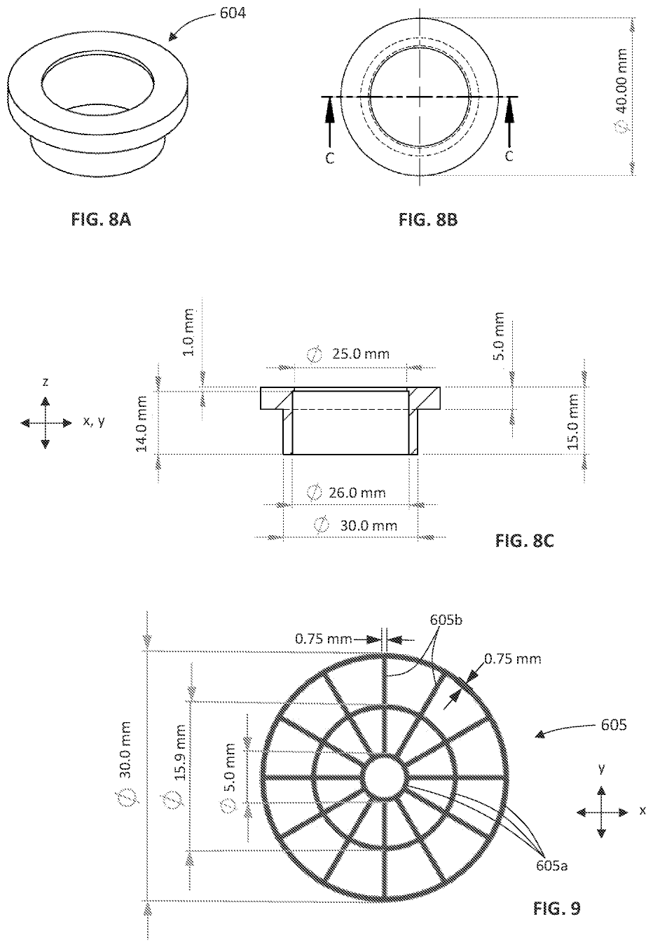

The measurement apparatus further includes a sample weight 604, which is machined of stainless steel to the configuration and dimensions shown in FIGS. 8A-8C. The small radially inwardly-projecting lip at the top portion of sample weight 604 is included for the purpose of providing a feature to grip to facilitate placement and removal of the sample weight 604 into and from the sample chamber.

The measurement apparatus further includes a sample support 605, which has the configuration and dimensions shown in FIG. 9. Sample support 605 has a z-direction caliper of 0.75 mm (which is its height when placed into position within the measurement apparatus in preparation for a measurement procedure). Each of the concentric ring portions 605a and radial spoke portions 605b of sample support 605 shown in FIG. 9 have an x-y plane width of 0.75 mm and a square cross section. Sample support 605 is configured to support a test specimen 616 within middle chamber portion 603b of base 603. Sample support 605 may be cut or machined from any material of suitable strength and corrosion resistance, such as, for example, brass sheet stock.

It will be noted that the outside diameter of sample support 605 and inside diameter of middle chamber portion 603b are both specified above to be 30.0 mm. Sample support 605 is disposed within middle chamber 603b during the measurement procedure. Accordingly, it will be appreciated that either or both of inside diameter of middle chamber portion 603b and outside diameter of sample support 605 may require slight adjustment to provide a small but sufficient clearance to enable sample support 605 to be conveniently inserted into and withdrawn from middle chamber portion 603b.

Similarly, it will be noted that the outside diameter of the lower portion of sample weight 604 and inside diameter of middle chamber portion 603b are both specified above to be 30.0 mm; and the outside diameter of the upper portion of sample weight 604 and inside diameter of upper chamber portion 603a are both specified to be 40.0 mm. The lower portion of sample weight 604 is disposed within middle chamber portion 603b, and the upper portion of sample weight 604 is disposed within upper chamber portion 603a, during the measurement procedure. Accordingly, it will be appreciated that either or both of inside diameter of middle chamber portion 603b and outside diameter of lower portion of sample weight 604, and either or both of inside diameter of upper chamber portion 603a and outside diameter of upper portion sample weight 604, may require slight adjustment to provide a small but sufficient clearance to enable sample weight 605 to be conveniently inserted into and withdrawn from middle chamber portion 603b.

The measurement apparatus further includes a computer (not shown) with suitable software and interfacing equipment configured to communicate with the valve actuator 610 to effect opening and closing of valve 607, and to receive and collect fluid height data from fluid height sensor 606 over time, at a rate of 100 Hz. The person of ordinary skill in the art will have sufficient knowledge and/or resources readily available to obtain components and configure the system including the computer and software to perform the operations described herein.

Test Fluid Preparation

The test fluid that is used for this measurement method is an aqueous solution of sodium chloride (NaCl) at a concentration of 0.9% by weight.

Components needed for the saline test fluid preparation include NaCl (reagent grade, CAS 7647-14-5) and deionized water. The NaCl is available from any convenient source, for example Sigma Aldrich item S9888.

The following preparation steps will result in approximately 2 liters of 0.9% wt NaCl test fluid: Add 18.0 g of NaCl to a 2 L Erlenmeyer flask, followed by the addition of 1982.0 g of deionized water. Stir until the NaCl is completely dissolved.

Measurement Procedure

To obtain a test specimen for measurement, lay a single layer of dry subject material out flat on a horizontal work surface, and die-cut a test specimen from it that is circular, with a diameter of 30 mm. Avoid areas of the material having folds, wrinkles or tears when selecting a location for sampling.

If the subject material is a layer component of an absorbent article (e.g., a feminine hygiene pad), for example, a topsheet or absorbent layer component, obtain a representative sample of the subject material that has not been incorporated into an absorbent article. Alternatively, if only fully manufactured absorbent articles are available as sources of the subject material, from an example thereof, separate the subject layer component from the article without stretching or damaging it. Once the subject layer component has been removed from the article, die-cut out a test specimen as described above. Precondition the test specimen at 23° C.±2 C.° and 50%±2% relative humidity for 2 hours prior to testing.

Referring to FIG. 7B, with the fluid valve 607 in closed position, insert the sample support 605 into the middle chamber portion 603b such that it lies horizontal/flat on the lower circumferential lip of middle chamber portion 603b. Using forceps, gently place the test specimen 616 over the sample support 605 so that it lays flat thereon, with no wrinkles. Now gently place the sample weight 604 over/onto the test specimen 616 such that the lower portion of the weight 604 is inserted into middle chamber portion 603b and rests on the test specimen about its circumferential edge, and the upper portion of the weight 604 is nested into the upper chamber portion 603a.

Now slowly add the previously prepared test solution to the fluid vessel 601, until an initial fluid surface 614 height Hi of 150 mm above the upper surface of the test specimen 616 is reached.

Allow the test specimen 616 to equilibrate within the filled sample chamber for about 60 seconds, and ensure there are no bubbles present on the surface of the test fluid or surface of the test specimen. If bubbles are present on the fluid surface, remove or pop them using a clean instrument. If bubbles are present on the upper surface of the test specimen 616, use a clean, round tip lab stirring rod to gently dislodge them, exercising care not to dislodge fibers (if the test specimen is fibrous), or stretch or damage the test specimen.

Secure the fluid height sensor 606 to the lid 602, and then place and fit the lid 602 over cylindrical wall 601a. Adjust the position of the fluid height sensor 606, if necessary, prior to the start of the test so as to prevent it from contacting the starting surface of the test fluid. Initially, the lower tip of the sensor 606 should be about 170 mm from the upper surface of the test specimen 616.

Position the collection vessel 613 below the valve 607.

Referring now to FIG. 7C, to start the measurement, simultaneously open the valve 607 and start the acquisition of decreasing fluid height Hd and time data to the nearest 0.01 mm and 0.01 seconds, respectively, with a data acquisition rate of 100 Hz. Test fluid will flow under gravitational pull through the sample chamber and through test specimen 616, sample support 605 and open valve 607, down into collection vessel 613, and test fluid surface 614 will fall while a surface 615 of collected fluid will rise. Height sensor 606 will sense and transmit data concerning the height of test fluid surface 614 at the designated sensing frequency, over time. The measurement is ended and the valve 607 is closed when test fluid has ceased exiting the valve, or after 1,000 seconds have elapsed, whichever occurs first. Remove the lid 602. Lift the sample weight 604 out of the sample chamber and remove it, and, using forceps, gently remove the wet test specimen 616 from the sample chamber, and proceed to measure the wet caliper of the test specimen.

The wet caliper of the test specimen 616 is measured promptly after completion of the measurement procedure, using a manually-operated micrometer equipped with a pressure foot capable of exerting a steady pressure of 2.07 kPa+0.07 kPa. The manually-operated micrometer is a dead-weight type instrument with readings accurate to 0.01 mm. A suitable instrument is Mitutoyo Series 543 ID-C Digimatic, available from Avantor/VWR International (Radnor, Pennsylvania) or equivalent. The pressure foot is a flat circular moveable face with a diameter of 19 mm. The test specimen is supported by a horizontal flat reference platform that is larger than and parallel to the surface of the pressure foot. Zero the micrometer against the horizontal flat reference platform. Transfer the wet test specimen 616 to the reference platform of the micrometer such that the specimen 616 is centered and lies horizontally and flat beneath the pressure foot. The pressure foot is lowered by hand with a descent rate of 3+1 mm/s until the full pressure (2.07 kPa) is applied to the test specimen. After 5 seconds elapse, the caliper of the wet test specimen is recorded as specimen caliper, to the nearest 0.01 mm. The test specimen is then discarded.

Remove test fluid inside the fluid vessel 601 and sample chamber, if any remains therein.

The procedure is repeated for a total of three replicate test specimens.

A separate “blank” run measurement is performed by following the procedure described above, but with only the sample support 605 and sample weight 604 present in the sample chamber (i.e. no test specimen is present). Note that the initial test fluid height Hi will be 150 mm above the upper surface of the sample support 605, rather than a surface of a specimen. This blank measurement will enable the permeability of the sample support 605 to be considered, when calculating the permeability of the test specimen.

Permeability Calculation

Total permeability, kₜₒₜₐₗ, is the permeability of the test specimen plus the sample support, calculated from the time and volume of flow through a fluid height decrease from 150 mm test fluid to 130 mm test fluid. Total permeability is calculated for each replicate test specimen using the following equation, and recorded to the nearest 0.01 E⁻¹⁰ m²:

$$ln ( \frac{Hi}{Hd} ) = \frac{{k}_{total} ρ g}{µ {L}_{total}} * \frac{{R}^{2}}{{r}^{2}} * t$$

$${k}_{total} = \frac{[ ( \frac{ln ( \frac{Hi}{Hd} )}{t} ) * \frac{{r}^{2}}{{R}^{2}} * µ * {L}_{total} ]}{ρ g} ;$$

thus, solved for kₜₒₜₐₗ:
where:
Hi=initial test fluid height (150 mm)
Hd=test fluid height as decreased at time t (for the present calculation, this is 130 mm)
t=time (seconds) elapsed when fluid height has decreased to 130 mm
kₜₒₜₐₗ=combined permeability of the test specimen and the sample support
p=density of the test fluid (kg/m 3)
g=gravitational constant (9.81 m/s²)=
viscosity of the test fluid (for purposes herein, assume 0.00109 kg/m-s)
Lₜₒₜₐₗ=combined caliper of the wet test specimen and the sample support (m)
R=the radius of the surface area of the test specimen through which the fluid flows ((26 mm/2)× (1m/1,000 mm)=0.013 m)
r=radius of the inside of the fluid vessel ((98.425 mm/2)×(1 m/1,000 mm)=0.049213 m)

The permeability of the sample support 605, kₛₛᵤₚ, is calculated in a similar manner, from the time and volume of flow through a fluid height decrease from 150 mm test fluid to 130 mm test fluid in the “blank” run. The permeability of the sample support 605 alone is described by the following equation, and recorded to the nearest 0.01 E⁻¹⁰ m²:

$$ln ( \frac{Hi}{Hd} ) = \frac{{k}_{ssup} ρ g}{µ {L}_{ssup}} * \frac{{R}^{2}}{{r}^{2}} * t$$

thus, solved for kₛₛᵤₚ:

$${k}_{ssup} = \frac{[ ( \frac{ln ( \frac{Hi}{Hd} )}{t} ) * \frac{{r}^{2}}{{R}^{2}} * µ * {L}_{frit} ]}{ρ g} ;$$

where:
Lssup=the caliper of the sample support 605 (0.00075 m)

The permeability of each replicate test specimen, k_{specimen}, is calculated from the following equation, then multiplied by 1.01324998 E⁺¹² and recorded to the nearest 0.1 Darcy:

$${k}_{specimen} = \frac{{L}_{specimen}}{( \frac{{L}_{total}}{{k}_{total}} ) - ( \frac{{L}_{ssup}}{{k}_{ssup}} )}$$

Now calculate the arithmetic mean of the test specimen permeability, k_{specimen}, across all three replicate test specimens, and report as Permeability to the nearest 0.1 Darcy.

Compression Recovery and Dry Caliper Measurement Methods

The compression recovery measurement method measures the compression recovery behavior along the z-direction of a test specimen, on a Constant Rate of Extension (CRE) universal mechanical test system (a suitable instrument is the MTS Alliance using TestSuite Software, as available from MTS Systems Corp., Eden Prairie, MN) using a load cell for which the forces measured are within 1% to 99% of the limit of the cell (preferably 100 N).

In the procedure, a sample of the subject material is slowly compressed along a z-direction to a maximum pressure of 3,446 Pa (0.5 psi), and then slowly released from compression. Its initial caliper at light contact compression of 39.79 Pa (0.0058 psi) is measured at the beginning of the loading portion of the cycle, and its final caliper at the same light contact compression is measured at the end of the unloading portion of the cycle. The final caliper divided by the initial caliper, multiplied×100%, is the compression recovery of the material for purposes herein.

All testing is performed in a room controlled at 23° C.±3C° and 50%±2% relative humidity, test specimens are conditioned in this environment for at least 2 hours prior to testing.

The upper and lower fixtures of the test system are circular parallel plate compression platens made of stainless steel. The platen mounted on the moveable CRE fixture has a diameter of 40 mm, and the platen mounted on the stationary CRE fixture has a diameter>40 mm. Both platens have adapters compatible with the mounts of the CRE test machine, capable of securing the platens with their opposing surfaces lying along parallel planes that are orthogonal to the motion of the crossbeam of the CRE test machine.

To obtain a test specimen for measurement, lay a single layer of dry subject material out flat on a horizontal work surface, and die-cut a test specimen from it that is circular, with a diameter of 40 mm. Avoid areas of the subject material having folds, wrinkles or tears when selecting a location for taking a specimen. If the subject material is a layer component of an absorbent article (e.g., a feminine hygiene pad), for example, a topsheet or absorbent layer component, obtain a representative sample of the subject material that has not been incorporated into an absorbent article. Alternatively, if only fully manufactured absorbent articles are available as sources of the subject material, from an example thereof, separate the subject layer component from the article without stretching or damaging it. Once the subject layer component has been removed from the article, die-cut out a test specimen as described above. Weigh the test specimen and record as dry mass to the nearest 0.001 g. A total of five test specimens are prepared. Precondition the test specimen at 23° C.±2 C.° and 50%±2% relative humidity for 2 hours prior to testing.

Prepare the universal test frame for a compression test to measure force and distance for one cycle of loading (compression) and unloading (recovery) as follows. The crosshead motion is programmed such that the upper platen moves down from a starting position with respect to the lower platen at a rate of 0.025 mm/s until an endpoint load of 4.33 N is reached (which applies endpoint pressure of 3,446 Pa (0.50 psi) between the platens) is reached, after which the crosshead movement is reversed and the upper platen is raised at the same rate of 0.025 mm/s, until the crosshead and platens are returned to the starting position. Adjust the platens such that the initial distance between their contact surfaces 25.0 mm (starting position), then zero the crosshead and load cell. Place the test specimen, with the wearer-facing surface upward, onto the lower platen, centered beneath the upper platen. Start the compression/release cycle and continuously collect force (N), time (s) and displacement (mm) data at a rate of 50 Hz.

Calculate the initial caliper of the test specimen by subtracting the crosshead displacement recorded, during the loading/compression portion of the cycle, at a light contact compression at 0.05 N force (39.79 Pa (0.0058 psi) pressure between the platens), from 25.0 mm, to the nearest 0.001 mm.

Calculate an interim caliper of the test specimen by subtracting the crosshead displacement recorded, during the loading/compression portion of the cycle, a compression at 0.8665 N force (689.5 Pa (0.10 psi) pressure between the platens), from 25.0 mm, to the nearest 0.001 mm. For purposes herein, this calculated value is the dry caliper of the specimen.

Calculate the final caliper of the test specimen by subtracting the crosshead displacement recorded, during the unloading/recovery portion of the cycle, at the light contact compression at 0.05 N force (39.79 Pa (0.0058 psi) pressure between the platens), from 25.0 mm, to the nearest 0.001 mm.

Calculate the compression recovery for the specimen as:

compression recovery=(final caliper/initial caliper)×100%

Repeat the procedure for each of the five test specimens, calculate the respective averages of the values obtained, and report the averages as Dry Caliper (average of the dry calipers for the five specimens) and Compression Recovery (average of the compression recoveries for the five specimens).

In view of the foregoing disclosure, the following examples are contemplated herein:

1. A feminine hygiene pad (10) comprising a liquid permeable topsheet (20), a liquid impermeable backsheet (40), and an absorbent system disposed between the topsheet and the backsheet, the absorbent system comprising:
an absorbent foam layer (30) beneath the topsheet, the foam layer having a wearer-facing surface and an outward-facing surface, and a plurality of apertures (31, 32) providing z-direction passageways from the wearer-facing surface to the outward-facing surface; and
a porous spacer layer (50) beneath the foam layer and underlying a majority, preferably substantially all, and more preferably all, of openings of the apertures in an outward-facing surface of foam layer.

2. The feminine hygiene pad of example 1 wherein the spacer layer (50) comprises a collection of fibers spun from one or more thermoplastic polymer resins (polymeric fibers).

3. The feminine hygiene pad example 2 wherein a majority, preferably substantially all, and more preferably all, of a basis weight of the fiber collection is constituted by the polymeric fibers.

4. The feminine hygiene pad of either of examples 2 or 3 wherein the polymer resins comprise one or more polymers selected from the group consisting of PE, PP and PET, and combinations thereof.

5. The feminine hygiene pad of any of examples 2-4 wherein the fibers are bicomponent fibers.

6. The feminine hygiene pad of example 5 wherein the bicomponent fibers are curled or crimped.

7. The feminine hygiene pad of either of examples 5 or 6 wherein the bicomponent fibers have a sheath-core configuration.

8. The feminine hygiene pad of example 7 wherein the core component comprises PET.

9. The feminine hygiene pad of either of examples 7 or 8 wherein the sheath component comprises PE.

10. The feminine hygiene pad of any of examples 2-9 wherein the polymer fibers have an average denier of at least 1, more preferably at least 2.

11. The feminine hygiene pad of any of the preceding examples wherein the spacer layer (50) is affixed within the pad via a deposit of adhesive.

12. The feminine hygiene pad of example 11 wherein the adhesive is disposed predominantly about a peripheral region (50p) of the spacer layer.

13. The feminine hygiene pad of example 12 wherein the adhesive is disposed predominantly between the spacer layer and the absorbent layer.

14. The feminine hygiene pad of example 11 wherein the adhesive is disposed predominantly between the spacer layer and the backsheet.

15. The feminine hygiene pad of any of the preceding examples wherein the spacer layer (50) has a void volume of at least 1,000 mm³.

16. The feminine hygiene pad of any of the preceding examples wherein the spacer layer (50) has a Permeability of at least 1,000 Darcys, preferably at least 3,000 Darcys, more preferably at least 5,000 Darcys.

17. The feminine hygiene pad of any of the preceding examples wherein the spacer layer (50) has a Dry Caliper no greater than 3.0 mm, more preferably no greater than 2.0 mm, even more preferably no greater than 1.5 mm, and still more preferably no greater than 1.0 mm.

18. The feminine hygiene pad of any of the preceding examples wherein the spacer layer (50) exhibits a Compression Recovery of at least 75 percent.

19. The feminine hygiene pad of any of the preceding examples wherein apertures present within a discharge region have an average x-y plane opening area at the wearer-facing surface of the absorbent layer of 3 mm² to 13 mm², more preferably 5 mm² to 10 mm².

20. The feminine hygiene pad of any of the preceding examples wherein apertures present within a discharge region have a numerical density of 3.0 to 9.0 apertures per cm², more preferably 4.0 to 8.0 apertures per cm², or even more preferably 5.0 to 7.0 apertures per cm², on the wearer-facing surface of the absorbent layer.

21. The feminine hygiene pad of any of the preceding examples wherein the foam layer (30) comprises a HIPE foam.

22. The feminine hygiene pad of example 21 wherein the foam layer (30) has two sublayers (30a, 30b) formed together, including a wearer-facing sublayer having a first average cell size and an outward-facing sublayer having a second average cell size, wherein the second average cell size is smaller than the first average cell size.

23. The feminine hygiene pad of any of the preceding examples wherein constituent material of the spacer layer (50) is hydrophilic or has been treated to render surfaces thereof hydrophilic. Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene pad comprising a liquid permeable topsheet, a liquid impermeable backsheet, and an absorbent system disposed between the topsheet and the backsheet, the absorbent system comprising:
an absorbent foam layer beneath the topsheet, the foam layer having a wearer-facing surface and an outward-facing surface, and a plurality of apertures providing z-direction passageways from the wearer-facing surface to the outward-facing surface; and
a porous spacer layer beneath the foam layer and underlying a majority, preferably substantially all, and more preferably all, of openings of the apertures in an outward-facing surface of foam layer.

2. The feminine hygiene pad of claim 1 wherein the spacer layer comprises a collection of fibers spun from one or more thermoplastic polymer resins (polymeric fibers).

3. The feminine hygiene pad claim 2 wherein a majority, preferably substantially all, and more preferably all, of a basis weight of the fiber collection is constituted by the polymeric fibers.

4. The feminine hygiene pad of claim 3 wherein the polymer resins comprise one or more polymers selected from the group consisting of PE, PP and PET, and combinations thereof.

5. The feminine hygiene pad of claim 4 wherein the fibers are bicomponent fibers.

6. The feminine hygiene pad of claim 5 wherein the bicomponent fibers are curled or crimped.

7. The feminine hygiene pad of claim 5 wherein the bicomponent fibers have a sheath-core configuration.

8. The feminine hygiene pad of claim 7 wherein the core component comprises PET.

9. The feminine hygiene pad of claim 7 wherein the sheath component comprises PE.

10. The feminine hygiene pad of claim 2 wherein the polymeric fibers have an average denier of at least 1.

11. The feminine hygiene pad of claim 1 wherein the spacer layer is affixed within the pad via a deposit of adhesive.

12. The feminine hygiene pad of claim 11 wherein the adhesive is disposed predominantly about a peripheral region of the spacer layer.

13. The feminine hygiene pad of claim 12 wherein the adhesive is disposed predominantly between the spacer layer and the absorbent layer.

14. The feminine hygiene pad of claim 1 wherein the spacer layer has a void volume of at least 1,000 mm³, and has a Dry Caliper no greater than 3.0 mm.

15. The feminine hygiene pad of claim 1 wherein the spacer layer has a Permeability of 1,000 Darcys to 6,000 Darcys.

16. The feminine hygiene pad of claim 1 wherein the spacer layer exhibits a Compression Recovery of at least 75 percent.

17. The feminine hygiene pad of claim 1 wherein apertures present within a discharge region have an average x-y plane opening area at the wearer-facing surface of the absorbent layer of 3 mm² to 13 mm².

18. The feminine hygiene pad of claim 17 wherein apertures present within a discharge region have a numerical density of 3.0 to 9.0 apertures per cm², on the wearer-facing surface of the absorbent layer.

19. The feminine hygiene pad of claim 1 wherein the foam layer comprises a HIPE foam.

20. The feminine hygiene pad of claim 1 wherein constituent material of the spacer layer is hydrophilic or has been treated to render surfaces thereof hydrophilic.
